(19) United States
(12) Patent Application Publication
(10) Pub. No.: US 2013/0149380 A1
(43) Pub. Date: Jun. 13, 2013

(54) USE OF DIATOMACEOUS EARTH IN THE PHARMACEUTICAL INDUSTRY

(71) Applicants: Endre Mikulásik, Kormend (HU); Ottó Albrecht, Budapest (HU)

(72) Inventors: Endre Mikulásik, Kormend (HU); Ottó Albrecht, Budapest (HU)

(73) Assignees: ONP HOLDINGS SE, Nicosia (CY); EGIS GYOGYSZERGYAR NYILVANOSAN MUKODO RESZVENYTARSASAG, Budapest (HU)

(21) Appl. No.: 13/700,605

(22) PCT Filed: May 27, 2011
(86) PCT No.: PCT/HU2011/000049

§ 371 Date: Feb. 20, 2013

(30) Foreign Application Priority Data
May 28, 2010 (HU) P1000278

(51) Int. Cl. A61K 47/02

(52) U.S. Cl. CPC A61K 47/02
USPC 424/474; 514/770; 424/400; 514/570; 514/567; 514/474; 424/94.21; 424/78.25; 424/637; 514/220; 514/763

(57) ABSTRACT

The present invention is related to solid pharmaceutical preparations containing diatomaceous earth (diatomite) or a natural mineral mixture containing diatomaceous earth as filler besides the active ingredient and optional other auxiliary agents. A further object of the invention is a method for manufacturing such pharmaceutical preparations.

### TECHNICAL FIELD OF THE INVENTION

The present invention pertains to the technical field of the formulation of pharmaceutical products and the excipients used therein. More closely, the present invention is related to solid pharmaceutical preparations containing diatomaceous earth or a natural mineral mixture containing diatomaceous earth as filler admixed with a solid pharmaceutically active ingredient and optional further auxiliary agents. The invention is also related to the use of diatomaceous earth as a filler in solid pharmaceutical dosage forms. A further object of the invention is a method for manufacturing pharmaceutical preparations containing diatomaceous earth or a natural mineral mixture containing diatomaceous earth.

### BACKGROUND OF THE INVENTION

Diatomaceous earth (siliceous earth, diatomite) is a sedimentary mineral mainly consisting of amorphous silicone dioxide originating from the shells of fossile dead diatomaceous algae (Diatoms). Besides the shells of the Diatoms, diatomaceous earth may contain further minerals (e.g. montmorillonite, caolinite, quartz, calcite, feldspar). Diatomaceous earth is a loamy, easily dispersible, small-grained, earthy, usually light-coloured silicaceous sedimentary mineral. It is of natural origin formed from the bulk deposition of the shells of fossile Diatoms in salty and freshwater lakes and seas of the early Jurassic period. Many occurances of the diatomaceous earth are known worldwide. It is produced by mining the natural deposit, separating and optionally physically or chemically treating the mineral. (Lloyd de Antonides 1998, Diatomite U.S. Geological Survey Mineral Commodity Summaries 1998; Tasnády Kubacska András: Ásványok). Diatomaceous earth has depositions in the United States (e.g. Nevada, California, Oregon, Washington), Canada, Germany, France, Demnark, the Czech Republic, Hungary. In Hungary, it is produced from the mines of Erdöbénye and Tállya.

Diatomaceous earth has been used in the industry for several purposes. The first significant invention involving diatomaceous earth was dinamite, which was discovered by Alfred Nobel in 1866. According to this invention, glycerol trinitrate was adsorbed onto diatomaceous earth, thus stabilizing glycerol trinitrate and obtaining a significantly more stable explosive which could be more conveniently transported and handled than the extremely sensitive liquid glycerol trinitrate.

In the industry, diatomaceous earth is being most widely used as a filtration aid. This use exploits the good porosity resulting from the particle shape of diatomaceous earth. Diatomaceous earth is industrially used as a filtering material, in filtration systems of swimming pools, in the purification of drinking water for the food industry and in the filtration of liquids, e.g. beer, wine and some syrups. Good filtration properties of diatomaceous earth are also utilized in paper industry, paint industry, textile industry and in the manufacturing of ceramics, soaps, washing powders and detergents. Diatomaceous earth is also used as a polishing agent in tooth pastes and in cleaning and polishing of metals due to its excellent abrasive properties.

Diatomaceous earth is heat-resistant, which renders it suitable for use in the manufacture of heat-resistant safety cabinets.

Diatomaceous earth is being used widely in the agriculture as an antiadhesive agent during the storage of grains and seeds. Diatomaceous earth is used as a mechanically acting insecticide. Fine particles of diatomaceous earth are absorbed in the exoskeleton of the insects and cause dehydration. Diatomaceous earth is used as vermicide for veterinary or human use. It is an important component of the nutrient media used in hydroponics systems due to its water-retaining property.

The adsorption properties of diatomaceous earth are utilized when applying the material as chromatographic sorbent during the separation of chemicals including DNA.

Due to its thermal inertness, diatomaceous earth can be used as a catalyst support. Published International Patent Application WO 2008127742 relates to a nanocatalyst, which is impregnated onto a porous material, i.e. diatomaceous earth.

Diatomaceous earth is used in the medicine in surgical instruments and as a modeling paste in dentistry.

Diatomaceous earth is also being widely used in the pharmaceutical industry. It is used as a silicon-containing active ingredient or an excipient in pharmaceutical formulations.

Diatomaceous earth is used as an active ingredient of commercial pharmaceutical compositions suitable for strenghtening the skeletal system, preventing osteoporosis, enhancing nail and hair formation and as cholesterol-lowering ingredient.

The use of diatomaceous earth as an excipient in pharmaceutical formulations has been described in several disclosures belonging to the state of the art.

Published International Patent Application WO2005004837 discloses a controlled release intravaginal drug-delivery system, wherein diatomaceous earth is used as a vehicle.

In Japanese Patent Application No. 01185267, the use of diatomaceous earth has been disclosed as a vehicle for volatile components and a liquid active ingredient in sprays intended for inhalation or for nasal use.

In Published International Patent Application No. WO 2008081539, the use of diatomaceous earth as a vehicle for Lactobacilli is disclosed, facilitating the transfer of said bacteria into the gastrointestinal tract.

Published International Patent Application No. WO 9917868 is related to a pharmaceutical preparation in form of a powder, wherein a liquid or a liquid mixture is absorbed into diatomaceous earth and transformed into a powdery form in this way.

Published International Patent Application No. WO98030640 discloses the utility of diatomaceous earth as superdisintegrant in the manufacture of pharmaceutical dosage forms, wherein the formulation is either to be desintegrated in the stomach or the absorption of the active ingredient from the stomach is intended.

Published US Patent Application No. 2010016448 describes the use of diatomaceous earth as disintegrant during the manufacture of orally disintegrating tablets.

The state of the art, however, is silent about solid pharmaceutical formulations wherein diatomaceous earth or a natural mineral mixture containing diatomaceous earth is used as filler (bulking agent) in solid pharmaceutical dosage forms admixed with a pharmaceutically active ingredient.

Fillers are a functional type of pharmaceutical excipients used in pharmaceutical preparations, which comprise the bulk of said preparation. While other auxiliary agents are used for adjusting the mode of action, stability, active ingredient release, organoleptic properties of the pharmaceutical preparation, the most important role of the filler resides in providing the consistent mass proportions and dose of the active ingredient as well as the uniform distribution and physical form and strength characteristic to the pharmaceutical unit dosage form, e.g. size, shape. Thus, the filler provides for transforming the active ingredient into a state suitable for administration.

Many pharmaceutical preparations contain only minute amounts (in some cases only milligrams or less) of the active ingredient, therefore the patient could not ingest the small amount of active ingredient or a great loss or irreproducibility of the administered dose could occur. For example, the weight of tablets can change between 100 mg and 1500 mg, while the weight of divisible tablets can vary between 300 mg and 2000 mg. Tablets developed for special purposes, e.g. chewable tablets or effervescent tablets can weigh up to 5000 mg. Such formulations are easy to ingest.

Sometimes the filler has another function in formulation as well, i.e. some fillers may function as disintegrants or moisture absorbing agents at the same time. Such properties are generally derivable from the physical-chemical properties of the substance in question.

Pharmaceutical preparations are usually produced in the form of unit dosage form. Such a unit dosage comprises a single dose of the active ingredient.

Many solid pharmaceutical formulations, such as tablets, dragees, film-coated tablets, pellets are produced by compression methods.

According to prior art, silicates and diatomaceous earth have been used as disintegrants. Disintegrants being another functional type of pharmaceutical excipients used in the proportion of 2-20% by weight in pharmaceutical formulations, are responsible for the disintegration of the dosage form and release of the active ingredient upon contact with moisture. This usually occurs shortly after ingestion in the mouth or in the stomach, i.e. usually within 5-15 seconds.

In the state of the art, diatomaceous earth is used as a vehicle for liquid or semisolid substances in pharmaceutical formulations. The liquid or semisolid active ingredient is distributed within and adsorbed onto diatomaceous earth acting as a solid support, usually transformed into a dosage form and the preparation thus obtained is administered to the patient. In such cases, diatomaceous earth provides for the the transformation of said liquid or semisolid active ingredient into solid adsorbed form more suitable for the operations of pharmaceutical technology for the manufacture of solid dosage form. Thus, function of the solid diatomaceous earth support is to provide the active ingredient with more favourable properties with regard to the formulation, rather than bulking the formulation, i.e. simply making up space.

In the pharmaceutical technology developed for the manufacture of compressed dosage forms, such as tablets or pellets, several chemically similar silicate compounds or silicate-containing minerals have been used for different purposes. Flocculated silicone dioxide is widely used in pharmaceuticals as a water adsorbing agent or a free-flow aid. Clay minerals like talc and caolin minerals are often applied in coatings as coating base and during tabletting as a lubricant and porosity-increasing agent. However, use of such agents is limited due to their extremely poor compressibility. Clay minerals and crystalline silicates are practically incompressible. Flocculated amorphous silicates are fluffy materials, which can not be used in greater proportions in the preparation. When a larger amount of an amorphous silicate has been used, the tablets exhibit exfoliation in thin layers and the tablets are deformed in a specific way. Among the presently known silicate compounds or minerals, none is suitable for the use as filler or bulking agent.

### SUMMARY OF THE INVENTION

The objective of our invention is providing pharmaceutical compositions, wherein the above-mentioned problems of formulating clay minerals, crystalline silicates and fluffy amorphous silicates have been solved by using a filler (bulking material) satisfying the requirements set forth by pharmaceutical technology requirements.

Surprisingly, we have found that the above-mentioned problems of formulation resulting from the disadvantegous properties of clay minerals, crystalline silicates and fluffy amorphous silicates can be solved by using natural diatomaceous earth as filler. Furthermore, we have surprisedly experienced that natural diatomaceous earth possesses several further advantageous properties, making it exceptionally suitable for the function of filler during the manufacture of pharmaceutical preparations. We have surprisingly found that despite of the disadvantages of clay minerals and silicates, diatomaceous earth exhibits excellent compressibility and can be compressed even in the absence of any other auxiliary tabletting agents. This recognition allows the use of diatomaceous earth in the manufacture of compressed pharmaceutical dosage forms, especially tablets and pellets.

Accordingly, an object of the present invention is a solid pharmaceutical preparation containing diatomaceous earth (siliceous earth, diatomite) or a natural mineral mixture containing diatomaceous earth, as filler in a mixture with the active ingredient and optional further excipients.

A further object of the invention is a method for manufacturing solid pharmaceutical preparations wherein diatomaceous earth (diatomite) or a natural mineral mixture containing diatomaceous earth is present as filler besides the active ingredient.

Another object of the present invention is the use of diatomaceous earth or a mineral mixture containing diatomaceous earth as filler in solid pharmaceutical formulations, most advantageously in formulations manufactured by compression.

### DETAILED DESCRIPTION OF THE INVENTION

In comparison with raw materials used for the purpose of fillers in pharmaceutical formulation operations, diatomaceous earth exhibits several advantageous properties, which render it extremely suitable for use as a filler in pharmaceutical formulations.

Despite of the poor compressibility of other siliceous minerals mentioned above, we have surprisingly found that diatomaceous earth can be compressed even in the absence of any other auxiliary tabletting agents. This recognition allows the use of diatomaceous earth in the manufacture of compressed pharmaceuticals dosage forms, especially tablets. Without being bound to the theory, the good compressibility of diatomaceous earth is believed to result from the comb-like interconnection of specially fossilated, sometimes partially broken Diatomite-husks. Compression can be carried out, however, by applying somewhat greater pressing force than that usually applied in formulation technology. Furthermore, the pliability of tablets is usually average. Although the pliability and strength of the compressed aggregate do not achieve those of the one obtained by using cellulose derivatives, it makes diatomaceous earth suitable for use in the compression of tablets. In the case when diatomaceous earth is compressed in admixture with a different, softer or less elastic material, particles of the second material are being forcefully intruded into the voids of diatomaceous earth particles, thus forming an adhesive bonding force which holds the ingredients in tablet form. Thus, in optimal cases, granulation occurs per se without the use of usual binders.

During our experiments we have found that diatomaceous earth can be used in similar amounts (2-98% by weight, preferably 20-80% by weight) as fillers known from the state of the art. In tablets, the proportion of diatomaceous earth can exceed 80% by weight and there is no limitation as to the upper limit for the proportion thereof.

Another distinct advantage of the diatomaceous earth used in pharmaceutical formulations according to the present invention resides in the fact that it is indifferent, it neither interacts with the active ingredient or auxiliary agents nor with the packaging material. Silicone dioxide comprises a predominant material of the Earth's crust, having exceptionally few reactions at a temperature tolerable for living organisms. Silicone dioxide reacts with other materials usually at a high temperature in the range of 600 to 1700° C. only, while reactions carried out in the pharmaceutical industry usually proceed under the temperature of 600° C. Silicone dioxide reacts during long-term exposition with concentrated acids or bases. Such conditions do not exist in pharmaceutical preparations since they are harmful to the human body. When packaged to provide air and humidity protections, diatomaceous earth preserves its chemical properties and quality for indefinite time, thus it is persistent and stable.

A special advantage of the use of diatomaceous earth as a filler resides in the fact that diatomaceous earth is easily wettable, which supports the appropriate distribution of the solvent (e.g. water, alcohols, glycols) during wet operations of pharmaceutical technology (e.g. during tablet manufacture, granulation, coating). Diatomaceous earth can be very easily wetted by water, aqueous granulating liquids, glycols, alcohols, organic solvents, oils and waxes. Wetting is an adsorptive process governed by capillary forces without the formation of chemical bonds. From the surface of diatomaceous earth, the wetting solvent can be rapidly and easily removed by drying. Diatomaceous earth withstands moisture and solvents, it is possible to adsorb a liquid of 140% weight of the weight thereof without any change of consistency.

A further advantage of diatomaceous earth is that it is biologically inert, rarely interacts with the human body and even if an interaction occurs, it is beneficial for the organism. Diatomaceous earth is not absorbed in the body and it is excreted in unchanged form. It is assumed that a very small amount of silicone is absorbed from certain amorphous silicates (silicic acid salts), which enhances the defensive ability of the body and nourishes the skin.

A further advantage is that diatomaceous earth is neither an allergen nor an hypersensibilizing agent. No example to such immune reactions is known from clinical practice, since dust allergy is usually caused by organic allergens found in the dust.

Diatomaceous earth is a naturally occuring mineral, which is persistent in the natural environment rather than having any natural cycle. Diatomaceous earth is not an environmentally polluting material, it does not pose any form of hazard, neither have any effect on the living environment. Surface properties of diatomaceous earth do not favour the proliferation of microorganisms. Due to its high water-adsorbing affinity, diatomaceous earth can even inhibit microbial life, thus it is unsuitable as a nutrient medium.

In summary, it can be concluded that the use of diatomaceous earth according to the present invention exhibits several properties which can be regarded as advatageous from the viewpoint of pharmaceutical industry: the natural mineral diatomaceous earth has suitable compressibility, good wettability, it is chemically indifferent, biologically inert, non-allergenic, inexpensive, durable, persistent and stable.

In comparison to diatomaceous earth, cellulose derivatives, being widely used in pharmaceutical industry, possess good compressibility, are inexpensive, chemically and biologically indifferent. However, during storage, physical properties of cellulose derivatives change, thus affecting the dissolution profile of the active ingredient. In the presence of humidity, cellulose derivatives undergo profound transformations due to physical and biological processes. Such processes do not occur in the case of diatomaceous earth, which is stable and easily wettable.

Compared to diatomaceous earth, starch derivatives exhibit excellent compressibility, they are inexpensive and of natural origin. However, starch derivatives are neither chemically, nor biologically indifferent. They are incompatible with humidity and can form a nutrient medium for polluting microorganisms, especially filamentous fungi and molds. Starch derivatives often influence dissolution. These processes have detrimental effects on stability.

Lactose has similar disadvantages to those of starch. An allergic reaction or intolerance to lactose is often encountered. According to some surveys, 20% of the population is slightly, 4% is severely sensitive to lactose (lactose intolerant).

Other substances suitable for the function of fillers are some inorganic carbonates or other inorganic salts, sugar alcohols or surface-active agents. However, the application area of these ingredients is limited due to the fact that they often interact with the active ingredient of the pharmaceutical composition.

The present invention is related to solid pharmaceutical preparations containing a pharmaceutically active ingredient admixed with diatomaceous earth or a natural mineral mixture containing diatomaceous earth, as filler, which may optionally contain further excipients.

More specifically, the present invention is related to solid pharmaceutical compositions containing a pharmaceutically active ingredient, a filler which comprises diatomaceous earth or a natural mineral mixture containing diatomaceous earth having 30-100% by weight, preferably more than 90% by weight of amorphous silicone dioxide content originating from diatomaceous algae. The natural mineral composition containing diatomaceous earth used as filler in the pharmaceutical composition according to the present invention usually consists of 30-100% by weight, preferably more than 90% by weight of amorphous silicone dioxide (calculated on the basis of dry matter) originating from diatomaceous algae, 0-30 weight montmorillonite, 0-30% by weight of calcite, 0-5% by weight of caolinite, 0-4% by weight of feldspar and 0-5% by weight of other minerals. The arsenic and lead content of the diatomaceous earth intended for use in pharmaceutical preparations according to the present invention is less than 10 mg/kg.

The particle size of the diatomaceous earth or that of a natural mineral mixture containing diatomaceous earth used in the pharmaceutical formulation according to the present invention as filler is in the range of 1 to 65 μm, preferably between 3 and 65 μm, the most advantageously 30-40 μm.

Moisture content of the diatomaceous earth used in the pharmaceutical compositions according to the present invention is within the range of 0-140%, usually between 0 and 40% by weight.

The quality of diatomaceous earth can be tested and controlled using methods known in the art. As an example, methods of the corresponding pharmacopoeial monographs can be used. Silicon dioxide content can be determined on the basis of the loss of weight upon reacting diatomaceous earth or a natural mineral mixture containing diatomaceous earth with hydrogen fluoride. Metal impurities, such as arsenic and especially heavy metals cadmium, lead and mercury can be assayed by atomic absorption spectroscopy. Amorphous or crystalline phases can be studied by X-ray diffraction analysis, while the shape of the particles containing the features of Diatomit husks can be examined by optical or electron microscopy.

The particle size of diatomaceous earth used as filler according to the present invention can be determined using laser beam diffraction. Such methods are included in pharmaceopoeia and used as industry standard (ISO-13320-1, Ph. Eur. 2.9.31.). A sample wetted with and dispersed in water can be used. Results are calculated according to the Mie theory, using general purpose, polydisperse model for irregularly shaped particles. Measurements can be carried out by any suitable instrument, such as Malvern MasterSizer 2000 analyzer and software.

The solid pharmaceutical preparation according to the present invention can be formulated in the pharmaceutical form of tablets, dragees, capsules, granules, pellets or in any other solid pharmaceutical dosage forms. Preferable pharmaceutical forms are manufactured by compression, such as tablets, pills, dragees or pellets.

A further object of the present invention is a method for the preparation of the pharmaceutical preparation according to the present invention, wherein said pharmaceutical preparation is manufactured by direct compression, by kneading and wet granulation, fluidized technology or by any other method suitable for the production of solid pharmaceutical dosage forms. Such techniques are known from the state of the art.

After mining and physical pretreatment, no further processing is required for the diatomaceous earth or the natural mineral mixture containing diatomaceous earth before use as filler in pharmaceutical products intended for human or veterinary use. However, the arsenic and lead content in a filler intended for the use in pharmaceutical formulations must not exceed 10 mg/kg.

During the purification and processing of the diatomaceous earth or material containing thereof intended for use as filler, methods known according to the art, e.g. milling, washing, calcination can be used with the limitation that the original structure, compressibility of the diatomaceous earth should be preserved. The method of the processing can be determined according to the intended use.

The proportion of amorphous silicone dioxide originating from diatomaceous algae or being present as diatomaceous earth is preferably at least 90% by weight calculated on the basis of dry matter in the filler used in pharmaceutical formulations according to the present invention.

Diatomaceous earth can be used without limitation with regard to the chemical structure of a pharmaceutically active ingredient during the formulation thereof. Thus, any solid active ingredients can be formulated using diatomaceous earth as filler.

The pharmaceutical formulation according to the present invention can contain any pharmaceutically active ingredient suitable for the treatment of the human or animal body. Such active ingredients include those which are suitable for the treatment, alleviation, prevention, diagnosis of diseases or pathological condition of the human or animal body. Furthermore, pharmaceutical formulations according to the present invention can contain an active ingredient suitable for determining the condition of the body or the mental status, restoration homeostasis, metabolism or substances produced otherwise by the body; conserving harmless microorganisms of the body; removing parasites or xenobiotics from the body, influencing the condition or function of the body or a member of the body or influencing mental status.

In the formulation of the present invention wherein diatomaceous earth is present as filler, any pharmaceutically active ingredients having therapeutical activity according to the above-mentioned definition and having suitable stability can be applied without limitation. Solid active ingredient can be preferably used in the form of particles.

Such pharmaceutically active ingredients are listed in encyclopedias, e.g. Merck Index, Rote Liste or Pharmindex.

Pharmaceutically active ingredients include those which are originating or obtained from living organisms or manufactured by biological processes excluding living organisms themselves.

Pharmaceutical formulations containing diatomaceous earth according to the present invention may contain, for example, an active ingredient belonging to the group of laxatives, analgesics, antidepressants, neuroleptics, sedatives, anxyolytics, antiinflammatory drugs, antibiotics, antivirals, anthelmintics, antiprotozoal agents, antimalarials, antirheumatics, antiallergics, histamine receptor antagonists, antiarrhytmics, antiepileptics, beta-receptor blockers, calcium-channel blockers, anticancer agents, enzymes, extracts, renin-angiotensin antagonists, broncholytics, antiasthmatics, diuretics, anti-gout agents, antidiabetics, immunsupressants, stimulants of the cardiovascular system, antihypertensives, antianginal agents, active agents used in the treatment of Alzheimer disease or Parkinson-disease, agents used in the treatment of osteoporosis, lipid-lowering agents, therapeutical agents suitable for the treatment of gastrointestinal or urological diseases, peptides, proteins, proton pump inhibitors, anticoagulants, active ingredients useful in the treatment of venous diseases, muscle relaxants, corticosteroids, sexual hormones, vitamines, minerals, amino or fatty acids.

The formulation according to the present invention, can contain, for example, acetaminophen, acyclovir, acetylcysteine, acetylcholin, alatrofloxacin, alendronate, algulcerase, alfuzosin, amantadine hydrochloride, ambenomium, amifostin, amiloride hydrochloride, aminocaproic acid, amphotericin B, human antihaemophilia factors, aprotinine, asparaginase, atenolol, atracurium besylate, atropine, azithromycin, aztreonam, BCG vaccine, bacitracin, becalermin, belladona, bepridyl hydrochloride, bleomycin sulfate, human calcitonin or salmon calcitonin, carboplatin, capecitabin, capreomycin sulfate, cefamandol, cefazolin, cefepime hydrochloride, cefixime, cefonicide, cefoperazone, cefotethan, cefotoxim, cefoxitin sodium, ceftizoxim, ceftriaxon, cefuroxim, cefalexin, cefapyrin sodium, cholera vaccine, gonadotropin, cidofovir, cisplatin, cladribin, clidinium bromide, clindamycin, ciprofloxacin, clondronate, colistimethate sodium, colistin sulfate, corticotropin, cosyntropin, cromalyn sodium, cytarabin, daltaperin sodium, danaproid, deferoxamine, denileukin diphtitox, desmopressin, diatrizoate meglumine or diatrizoate sodium, dicyclomine, didanozin, dirithromycin, dopamine hydrochloride, dornase alfa, doxacurium chloride, doxorubicin, editronate disodium, elanaflate, encephalin; enoxacin; ephedrine; epinephrine; erithropoetin; erithromycin; esmol hydrochloride; factor IX; famicyclovir; fludarabin; fluoxetin; foscarnet sodium; gancyclovir; granulocyte colony stimulating factor or a derivative thereof; granulocyte-macrophage stimulating factor; human or bovine growth factor, gentamycin; glucagon; glykopyrolate; gonadotropin releasing hormone and synthetic analogs thereof, gonadorelin; grepafloxacine; hemophylic B vaccine; hepatitis A vaccine; hepatitis B virus vaccine; heparin; indinavir sulfate; influenza virus vaccine; interleukin-2; interleukin-3; human insulin; interferon alfa; interferon beta; ipratropium bromid; isofosfamide; encephalitis virus vaccine; lamivudin; leucovorin calcium; leuprolide acetate; levofloxacin; lincomycin; lobucavir; lomefloxacin; loracarbef; mannitol; measles virus vaccine; meningococcus vaccine; menotropins; mefensolate bromide; mesalmin; misolastin; methanamine; methothrexate; metscopolamine; metformine hydrochloride; metoprolol; mezocyllin sodium; mivacurium chloride; mumps virus vaccine; nedocromil sodium; neostigmin bromide; neostigmin methyl sulfate; neutontine; norfloxacin; octreotide acetate; ofloxacin; olpadronate; oxytocin; pamidronate disodium; pancuronium bromide; paroxetine; pefloxacine; pentamindine isothionate; pentostatine; pentoxiphylline; pericyclovir; pentagastrine; phentolamine mesylate; phenylalanine; physostigmin salicylate; plague vaccine; piperacilline sodium; pneumococcus vaccine; poliovirus vaccine; polymixin B sulfate; pralidoxine chloride; pramlintide; pregabalin; propofenon; propenthaline bromide; pyridostigmine bromide; rabies vaccine; risedronate; ribavarine; rimantadin hydrochloride; rotavirus vaccine; salmeterol xinafoate; sincalide; sotalol; somatostatin; sparfloxacin; spectinomycin; stavudine; streptokinase; streptozocin; suxamethonium chloride; tacrin hydrochloride; terbutalin sulfate; thiopeta; ticarcilline; tiludronate; timolol; tissue plasminogen activator; TNFR:Fc; TNK-tPA; trandolapril; trimetrexate gluconate; trospectinomycin; trovafloxacin; tubocurarin chloride; tumor necrosis factor; typhoid vaccine; urea; urokinase; vancomycin; valacyclovir; valsartan; varicella virus vaccine; vasopressin and derivatives thereof; vecoronium bromide; vinblastin; vincristin; vinorelbin; vitamin B 12; warfarin sodium; yellow fever vaccine; zalcitabin; zanamavir; zolendronate; zidovudin; aminoglutethimide, amiodaron, amlodipine, amphetamine, amphotericin B, atorvastatin, atovaquone, azithromycin, baclofen, beclomethazon, benazepril, benzonatat, betamethasone, bicalutamide, budesonide, bupropion, busulfan, butenafm, calcifediol, calcipotrien, calcitriol, camptothecin, candesartan, capsaicine, carbamazepine, carotinoids, celecoxib, cerivastatin, cetirizin, chlorpheniramine, cholecalciferol, cilostazol, cimetidin, cinnarizine, ciprofloxacin, cisapride, clarithromycine, clemastine, clomiphene, clomipramine, clonazepam, clopidogrel, codeine, coenzym Q10, cyclobenzaprine, cyclosporin, danazole, dantrolene, dexchlorpheniramine, diazepam, diclofenac, dicoumarol, digoxin, dehydroepiandrosteron, dihydroergotamine, dihydrotachisterol, diritromycine, donezepil, efavirenz, eposartan, ergocalciferol, ergotamine, essential fatty acids, etodolac, etoposide, famotidin, fenofibrate, phentanyl, fexofenadine, fmasteride, fluconazole, flurbiprofen, fluvastatin, fosfenytoin, frovatriptan, furasolidon, gabapentin, gemfibrozil, glibenclamide, glipizide, glyburide, glimepiride, griseofulvin, halophantrin, hydrochlorothiazide, ibuprofen, irbesartan, irinotecan, isosorbide dinitrate, isothretinoin, itraconazole, ivermectin, ketoconazole, ketorolac, lamotrigine, lansoprazole, leflunomide, lisinopril, loperamide, loratadine, lorazepam, lovastatin, L-thyroxine, lutein, medroxyprogesterone, mifepriston, mefloquin, megestrol acetate, methadone, methoxsalen, metronidazole, miconazole, midazolam, miglitol, minoxidil, mitoxanthron, montelukast, nabumeton, nalbufin, naratriptan, nelfmavir, nifedipine, nilsolidipine, nilutanide, nitrofurantoin, nizatidine, omeprazole, oprevelkin, estradiol, oxaprosin, paclitaxel, paracalcitol, paroxetine, pentazocine, pioglitazone, pizofetine, pravastatin, prednisolone, probucol, progesterone, pseudoephedrine, pyridostigmine, rabeprazole, raloxifene, rofecoxib, repaglinide, rifabutin, rifapentine, rimexolone, ritanovir, rizatriptan, rosiglitazone, saquinavir, sertraline, sibutramine, sildenafil, simvastatin, sirolimus, spironolactone, sumatriptan, tacrine, tacrolimus, tamoxifen, tamsulosin, targretin, tazaroten, telmisartan, teniposide, terbinafine, terazosin, terbutaline tetrahydrocannabinol, tiagabin, ticlopidine, tirofibran, tisanidin, topiramate, topotecan, toremifen, tramadol, tretinoin, troglitazone, trovafloxacin, ubidecarenon, valsartan, venlafaxin, verteporfm, vigabatrin, vitamin A, vitamin D, vitamin E, vitamin K or a derivative thereof, zafirlukast, zileuton, zolmitriptan, zolpidem, zopiclon, cytokines, peptidomimetics, peptides, proteins, antibodies, vaccines, nucleosides, nucleotides, nucleic acids, vitamins such as carotinoids, vitamin E, vitamin D, vitamin C, thiamine, riboflavine, niacin, folic acid, pyridoxine, biotin, pantothenic acid, cyanocobalamine; minerals, e.g. magnesium, manganese, zinc, selenium, chromium, copper, food supplements including but not limited to alfa lipoic acid, lutein, beta-carotinoids.

Active ingredients can be present in form of their pharmaceutically acceptable salts or other derivatives in the pharmaceutical formulations according to the present invention, such as in case of chiral forms of active ingredients, in the form of the optically active isomers, the racemic compounds, diastereomers or mixtures thereof; chlathrates, chelates, complexes, polymorphs or prodrugs of the active ingredient. The pharmaceutical formulation according to the present invention can contain two or more active ingredients.

By using diatomaceous earth according to the present invention, several problems and objectives arising during the pharmaceutical technology of solid dosage forms can be solved.

Diatomaceous earth or a natural mineral mixture containing thereof can be used in the pharmaceutical formulation according to the present invention in a proportion between 2-98% by weight, advantageously between 20 and 80% by weight.

Use of diatomaceous earth renders possible the formulation of tablets having very high (weight of the active ingredient is 50% by weight or more related to the tablet weight) or very low (the weight of the active ingredient is 0.5% by weight or less related to the tablet weight) content of active ingredient.

In case of solid formulations having very low content of active ingredient, said active ingredient can be excellently distributed in highly disperse inert diatomaceous earth. Since a large proportion of diatomaceous earth consists of amorphous material, only a few active sites are present on its surface, therefore the active ingredient resides on said surface as an amorphous layer rather as crystals. Dissolution of such formulations in the stomach occurs rapidly, therefore its bioavailability can be much higher than that of a preparation containing crystalline active ingredients.

Diatomaceous earth can be used in solid formulations containing a moisture-sensitive active ingredient. Since diatomaceous earth acts as a moisture-absorbing filler, thus decreases the rate of moisture-related decomposition of the active ingredient.

In solid formulations containing a hearburn-causing active ingredient, diatomaceous earth of high dispersity can act as an indifferent filler not impaired by metal salts. Metal salts dried onto the surface of diatomaceous earth can contact the stomach wall at a great area, thereby the risk of erosion and discomfort after the ingestion of the medicament is significantly reduced. In case of formulations wherein the active ingredient has viscous liquid physical state, diatomaceous earth acts as an adsorptive filler preventing the oily spotting of the surface of the formulation.

The following type examples demonstrate the composition and manufacturing methods of the pharmaceutical formulation according to the present invention without limiting the scope of protection to said examples. Throughout the examples, diatomaceous earth obtained in Erdöbénye, Hungary has been used. However, in the pharmaceutical formulations according to the present invention, diatomaceous earth obtained from any geographical location can be used.

### EXAMPLE 1

### Tablets Manufactured by Direct Compression Having High Content of Active Ingredient

Ibuprofen Tablets of 500 mg Strength

Composition:

| | | |
|---|---|---|
| Ibuprofen (DC) | 500 | mg |
| Collidon 30 | 15 | mg |
| Diatomaceous earth | 80 | mg |
| Magnesium-stearate | 5 | mg |

Manufacturing Method (Laboratory Scale):

The ingredients are homogenized manually and tablets are compressed using a 13-mm, biplanar tool.

Apparatus: Riva Piccola Type B/D4 tabletting machine.

Compression force: 18 kN

### EXAMPLE 2

### Tablets Manufactured by Wet Granulation Having High Content of Active Ingredient

Methyldopa Tablets of 500 mg Strength

Composition

| | | |
|---|---|---|
| Methyldopa | 500 | mg |
| Diatomaceous earth (portion I). | 200 | mg |
| Kollidon 30 | 30 | mg |
| Ethanol | 100 | mg |
| Diatomaceous earth (portion II). | 50 | mg |
| Magnesium-stearate | 5 | mg |

Manufacturing Method (Laboratory Scale):

Methyldopa and portion I of diatomaceous earth are homogenized manually. Kollidon 30 is dissolved in ethanol and kneaded manually with the mixture of the active ingredient and the first portion of diatomaceous earth. The mass thus obtained is spread out onto a tray and dried at room temperature. Subsequently the mixture is regranulated using a 0.8 mm sieve, mixed with the second (II) portion of diatomaceous earth and the magnesium stearate and compressed into tablets using a 13-mm diameter biplanar tool.

Apparatus: Kilian RTS 21D tabletting machine.

Compression force: 60 kN

### EXAMPLE 3

### Tablets Manufactured by Direct Compression, Containing a Moisture-Sensitive Active Ingredient

Ascorbic Acid Tablets of 100 mg Strength

Diatomaceous earth acts in this composition as a filler-desiccant, thus significantly reducing the rate of decomposition of the active ingredient.

Composition:

| | | |
|---|---|---|
| Ascorbic acid DC | 100 | mg |
| Diatomaceous earth | 137 | mg |
| HPMC DC | 10 | mg |
| Magnesium-stearate | 3 | mg |

Manufacture (Laboratory Scale):

Ascorbic acid is mixed with hydroxypropymethylcellulose and diatomaceous earth, homogenized manually and compressed into tablets using a 10 mm biplanar tool.

Equipment: Kilian RTS 21D tabletting machine

Compression force: compaction (stage) force: 20 kN,

compression (main) force: 100 kN

### EXAMPLE 4

### Enzyme-Containing Tablets Manufactured by Direct Compression

In this composition, diatomaceous earth acts as a desiccant filler, thus extending the shelf life of the enzyme.

Pancreatin Tablets 100 mg

Composition:

| | | |
|---|---|---|
| Pancreatine | 100 | mg |
| Avicel PH 101 | 50 | mg |
| Diatomaceous earth | 95 | mg |
| Magnesium stearate | 5 | mg |

Manufacture (Laboratory Scale):

Pancreatine is mixed with the other excipients, thereafter diatomaceous earth is added, mixed and homogenized manually. Tablets are pressed using a 10-mm biplanar tooling.

Apparatus: Kilian Type RTS 21D tabletting machine

Compression force: compaction (stage) force 20 kN,

compression (main) force: 100 kN

### EXAMPLE 5

### Dispersive Tablet Manufactured by Direct Compression Containing a Moisture-Sensitive Active Ingredient,

In this composition, PVP-iodine forming a sticky mass in the presence of water is dispersed in diatomaceous earth. The composition is free from added binder. Diatomaceous earth acts as a wetting agent during dissolution as well, thus assisting the quick dispersion of the tablet.

PVP-Iodine Tablets 100 mg

Composition:

| | | |
|---|---|---|
| PVP-iodine | 100 | mg |
| Diatomaceous earth | 145 | mg |
| Magnesium stearate | 5 | mg |

Manufacture (Laboratory Scale):

The PVP-iodine is mixed with the diatomaceous earth and homogenized manually. Tablets of flat surface are compressed using 12-mm biplanar tooling. Before use, the tablets are added to water, thus producing a disinfectant liquid.

Apparatus: Riva Piccola Type B/D4 tabletting machine.

Compression force: 18 kN

### EXAMPLE 6

### Tablets Containing a Stomach-Irrritating Active Ingredient, Manufactured by Fluidization

Diatomaceous earth acts in this composition as a highly disperse indifferent filler compatible with metal salts. Furthermore, using diatomaceous earth allows that metal salts contact the stomach wall at a great area, thus decreasing the risk of erosion and patient discomfort after administration.

Iron (II)-Sulfate (65 mg)+Manganese(II)-Sulfate (3.5 mg)+Copper(II)-Sulfate (0.16 mg) Tablets

Composition:

| | | |
|---|---|---|
| Iron (II) sulfate | 65 | mg |
| Manganese-sulfate | 3.5 | mg |
| Copper-sulfate | 0.16 | mg |
| Diatomaceous earth | 250 | mg |
| Purified water | 200 | mg |
| Ethanol | 100 | mg |
| Kollidon 30 | 30 | mg |
| Magnesium stearate | 2 | mg |

Manufacture (Laboratory Scale)

The iron(II)-sulfate, manganese(II)-sulfate and copper(II)-sulfate are dissolved in purified water. Kollidone 30 is dissolved in ethanol. The diatomaceous earth is placed into a fluid apparatus and the solution of metal salts is sprayed onto the fluidized bed. The powder is dried in an air flow, subsequently transformed into granules by spraying the alcoholic collidone solution. The granules are dried in an air stream and regranulated using an 1-mm sieve. The magnesium stearate is added and homogenized manually. Tablets are pressed using 12-mm biplanar tooling.

Apparatus:

FPG-2 rotating fluidizing apparatus:
Inlet air temperature: 50° C.
   Fluidizing air flow: 10-25 m³/hour, depending on the wetting of the bed (automatic control)
Spraying air flow: 0.3 m3/hour
Spraying pressure: 0.6 bar
Liquid flow rate: 15 ml/min

Apparatus: Kilian Type RTS 21D tabletting machine.

Compression force: compaction (stage) force: 20 kN,

compression (main) force: 80 kN

### EXAMPLE 7

### Tablets Manufactured by Fluid Technology Having Low Content of Active Ingredient

Diatomaceous earth acts as a highly disperse inert filler allowing formulation of extremely small amount of an active ingredient. Diatomaceous earth consists mainly an amorphous component (silicone dioxide), which contains only a few active sorption sites. Thus, the active ingredient is postulated to be present on the surface of the amorphous filler as an amorphous layer rather than in crystalline form. Dissolution of such formulations in the stomach is very quick and in some cases the bioavailability exceeds that of the formulations containing the active ingredient as crystals.

Alprazolam Tablets 0.25 mg

Composition:

| | | |
|---|---|---|
| Alprazolam | 0.25 | mg |
| Diatomaceous earth | 200 | mg |
| Ethanol | 100 | mg |
| Avicel PH 101 | 95 | mg |
| Magnesium stearate | 5 | mg |

Manufacture (Laboratory Scale):

Aprazolam is dissolved in the ethanol. The diatomaceous earth is placed into the fluid bed apparatus and the ethanolic solution of alprazolam is sprayed thereon. The powder is dried and regranulated using a 0.8 mm sieve. Avicel PH 101 and magnesium stearate are added and homogenized manually. Tablets are pressed using 10-mm biplanar tooling.

Apparatus:

FPG-2 rotating fluidizing apparatus
Inlet air temperature 45° C.
   Fluidizing air flow: 10-25 m³/hour, depending on the wetting of the fluid bed (automatic control)
Spraying air flow: 0.3 m³/hour
Spray pressure: 0.6 bar
Spray liquid flow rate: 5 ml/min

Apparatus:: Kilian Type RTS 21D tabletting machine.

Compression force: compaction (stage) force: 20 kN,

compression (main) force: 60 kN

### EXAMPLE 8

### Tablets Containing an Oily Active Ingredient, Manufactured by Direct Compression

Diatomaceous earth acts in this composition as an adsorptive filler binding the active ingredient having oily surface. In this way, spotting of the surface of the tablets and other esthetic defects can be prevented.

Beta-Carotene Tablets 50 mg

Composition:

| | | |
|---|---|---|
| Beta-carotene (in form of a 10% powder) | 500 | mg |
| Diatomaceous earth | 340 | mg |
| Magnesium stearate | 10 | mg |

Manufacture (Laboratory Scale):

The components are blended manually, thereafter tablets are pressed using a 13-mm diameter biplanar tooling.

Apparatus: Kilian Type RTS 21D Tabletting machine.

Compression force: compaction (stage) force: 20 kN,

compression (main) force: 80 kN

1. A solid pharmaceutical formulation containing an active ingredient, diatomaceous earth or a natural mineral mixture containing diatomaceous earth as filler and optional further pharmaceutically acceptable excipients.

2. A solid pharmaceutical formulation according to claim 1 produced by compression.

3. A solid pharmaceutical formulation according to claim 1 presented as unit dosage form.

4. A solid pharmaceutical formulation according to claim 1, wherein the diatomaceous earth or the natural mineral mixture containing diatomaceous earth used as filler contains 30-100% by weight, preferably more than 60% by weight, the most advantageously more than 90% by weight of amorphous silicon dioxide originating from diatomaceous algae.

5. A solid pharmaceutical formulation according to claim 1, wherein the natural mineral mixture containing diatomaceous earth being used as filler contains 30-100% by weight, preferable more than 60% by weight, the most advantageously more than 90% by weight of amorphous silicone dioxide originating from diatomaceous algae, 0-30% by weight of montmorillonite, 0-30% by weight of calcite, 0-5% by weight of caolinite, 0-3% by weight of quartz, 0-4% by weight of feldspar and optionally 0-5% by weight of other minerals.

6. A solid pharmaceutical formulation according to claim 1, characterized in that the arsenic content of the filler comprising diatomaceous earth or diatomaceous earth-containing natural mineral mixture intended “for use in humans is less than 10 mg/kg.

7. A solid pharmaceutical formulation according to claim 1, characterized in that the lead content of the filler comprising diatomaceous earth or diatomaceous earth-containing natural mineral mixture intended for use in humans is less than 10 mg/kg.

8. A solid pharmaceutical formulation according to claim 1, characterized in that the particle size of the diatomaceous earth or natural mineral mixture containing diatomaceous earth used in said formulation as filler is between 1 and 65 μm, preferably between 3 and 65 μm, the most advantageously between 30 and 40 μm.

9. A solid pharmaceutical formulation according to claim 1 containing a solid pharmaceutically active ingredient.

10. A solid pharmaceutical formulation according to claim 1, presented in the form of tablet, film-coated tablet, dragee, capsule, granule, pellet or any other solid pharmaceutical dosage form.

11. A pharmaceutical formulation according to claim 1, wherein the proportion of the diatomaceous earth or natural mineral mixture containing diatomaceous earth is between 2-98% by weight, advantageously between 20 and 80% by weight.

12. A method for the manufacture of a solid pharmaceutical formulation according to claim 1, which comprises admixing diatomaceous earth and the pharmaceutically active ingredient, optionally carrying out further pharmaceutical operations and producing the solid pharmaceutical formulation according to claim 1 by direct compression, wet kneading and compression or fluid-bed granulation.

13. (canceled)
